(19) United States
(12) Patent Application Publication
(10) Pub. No.: US 2024/0206765 A1
(43) Pub. Date: Jun. 27, 2024

(54) Photogrammetric measurement of body dimensions using patterned garments

(71) Applicant: LIKE A GLOVE LTD., Petah Tikva (IL)

(72) Inventors: Simon Cooper, Ashdod (IL); Michael Slobodkin, Maple (CA)

(21) Appl. No.: 18/556,685

(22) PCT Filed: Oct. 5, 2022
(86) PCT No.: PCT/IB2022/054306

§ 371 Date: Oct. 23, 2023

Related U.S. Application Data
(63) Continuation-in-part of application No. PCT/IB2020/052526, filed on Mar. 19, 2020.
(60) Provisional application No. 63/187,435, filed on May 12, 2021.
(60) Provisional application No. 62/937,265, filed on Nov. 19, 2019.
(60) Provisional application No. 62/939,730, filed on Nov. 25, 2019.

(51) Int. Cl. A61B 5/107; A41D 1/00; A61B 5/00; G06T 7/80; H04N 23/50

(52) U.S. Cl. CPC A61B 5/1075; A41D 1/002; A61B 5/0077; A61B 5/743; G06T 7/80; H04N 23/50

(57) ABSTRACT

Measurement apparatus includes a garment (24, 26) including an elastic fabric (27) having a predefined pattern (28) extending across a surface thereof and configured to be worn over a part of a body of a subject, such that the elastic fabric stretches across the part of the body. An imaging module (30) includes a fastener (40) configured for attachment to a mobile electronic device (32) having a camera module and a conical extension (41) having a rear opening (46), which is configured to be held by the fastener in alignment with the camera module, and a front opening (48), which is larger than the rear opening and is configured to contact and traverse across the fabric. The camera module captures through the front opening images that are indicative of local deformations of the pattern of the fabric.

### CROSS-REFERENCE TO RELATED APPLICATION

This application claims the benefit of U.S. Provisional Patent Application 63/187,435, filed May 12, 2021. This application is also a continuation in part of PCT Patent Application PCT/IB2020/052526, filed Mar. 19, 2020, which claims the benefit of U.S. Provisional Patent Application 62/937,265, filed Nov. 19, 2019, and of U.S. Provisional Patent Application 62/939,730, filed Nov. 25, 2019. All of these related applications are incorporated herein by reference.

### FIELD OF THE INVENTION

The present invention relates generally to measurement of body size and shape, and particularly to apparatus and methods for automating such measurements.

### BACKGROUND

Accurate measurement of the size and shape of a human body can be useful in a number of applications. Such measurements can be used, for instance, in identifying and purchasing articles of clothing that will fit a particular customer, as well as tracking changes in body proportions and shape for purposes of health and fitness monitoring.

As one example of this sort of application, PCT International Publication WO 2015/181661, whose disclosure is incorporated herein by reference, describes measurement apparatus, including an elastic fabric, configured as a garment to be worn over a part of a body of a human subject. One or more conductive fibers are integrated with the elastic fabric to as to stretch together with the elastic fabric when worn over the part of the body. A controller is coupled to measure an electrical property of the one or more conductive fibers in response to stretching of the elastic fabric, and to output an indication of a dimension of the part of the body based on the measured property.

Other systems extract body size measurements by processing images of the body. For example, U.S. Pat. No. 8,908,928 describes methods and systems for generating a size measurement of a body part of person for fitting a garment. The methods include providing photographic data that includes images of the body part and using feature extraction techniques to create a computer model of the body part.

### SUMMARY

Embodiments of the present invention that are described hereinbelow provide improved methods, systems, and garments for use in measuring body dimensions.

There is therefore provided, in accordance with an embodiment of the invention, measurement apparatus, including a garment, which includes an elastic fabric having a predefined pattern extending across a surface thereof and configured to be worn over a part of a body of a subject, such that the elastic fabric stretches across the part of the body. An imaging module includes a fastener configured for attachment to a mobile electronic device having a camera module, and a conical extension having a rear opening, which is configured to be held by the fastener in alignment with the camera module, and a front opening, which is larger than the rear opening and is configured to contact and traverse across the fabric, whereby the camera module captures through the front opening images that are indicative of local deformations of the pattern of the fabric.

In some embodiments, the apparatus includes a processor, which is configured to process the images captured by the camera module at multiple locations on the surface of the fabric so as measure a local deformation of the pattern at the multiple locations due to stretching of the fabric, and to compute a dimension of the part of the body responsively to the measured deformation. In a disclosed embodiment, the processor is contained in the mobile electronic device.

Additionally or alternatively, the apparatus includes a cap, having a calibration pattern formed thereon, which is configured to fit over the front opening of the conical extension, wherein the processor is configured to calibrate a magnification of the camera by processing an image of the calibration pattern captured by the camera module and to apply the calibrated magnification in measuring the local deformation of the pattern.

Typically, the processor is configured to output an indication of the computed dimension to a user of the apparatus.

In a disclosed embodiment, the mobile electronic device is a smartphone, and the fastener includes a housing configured to fit over the smartphone.

In some embodiments, the pattern includes multiple graphical symbols, which are disposed at different, respective locations across the surface of the fabric and encode information identifying the respective locations.

In some embodiments, the imaging module includes a transparent window covering the front opening of the conical extension so as to contact the surface of the fabric. In a disclosed embodiment, the window is oriented at an angle that is not parallel to a plane of the rear opening of the conical extension.

There is also provided, in accordance with an embodiment of the invention, an imaging module, including a housing configured to fit over a smartphone and a conical extension having, at a rear end of the conical extension, a rear opening, which is configured to be held by the housing in alignment with a camera module of the smartphone, and having, at a front end of the conical extension, opposite the rear end, a front opening, which is larger than the rear opening.

In a disclosed embodiment, the module includes a locking connection, which attaches the conical extension to the housing. Alternatively, the conical extension is permanently fixed to the housing.

There is additionally provided, in accordance with an embodiment of the invention, a method for measurement, which includes providing a garment including an elastic fabric having a predefined pattern extending across a surface thereof and configured to be worn over a part of a body of a subject, such that the elastic fabric stretches across the part of the body. While the subject is wearing the garment, images of the pattern are captured using a camera, while the camera contacts and traverses across the surface of the fabric. The images captured by the camera at multiple locations on the surface of the fabric are processed so as measure a local deformation of the pattern at the multiple locations due to stretching of the fabric. An indication of the locations at which the images were captured is output to a user.

In a disclosed embodiment, the method includes computing a dimension of the part of the body responsively to the measured deformation, and outputting an indication of the computed dimension to the user.

In some embodiments, processing the images includes tracking the locations on the surface of the fabric at which the camera captures the images, and outputting the indication includes prompting a user, responsively to the tracked locations, to shift the camera to an area of the garment in which the images of the pattern have not yet been captured. In one embodiment, prompting the user includes directing the user to shift the camera so as to traverse at least some of the locations multiple times, whereby the camera captures at least first and second images of each of the at least some of the locations, and processing the images includes comparing the first and second images so as to verify an accuracy of measurement of the local deformations. Typically, directing the user includes presenting the locations on a display, and marking the locations on the display to indicate a number of times that each of the locations has been traversed.

In another embodiment, capturing the images includes illuminating an area contacted by the camera, and the method includes capturing one or more image frames while the area is not illuminated, and verifying that the one or more image frames are dark as an indication that the camera is in contact with the surface of the fabric.

The present invention will be more fully understood from the following detailed description of the embodiments thereof, taken together with the drawings in which:

### BRIEF DESCRIPTION OF THE DRAWINGS

FIG. 1 is a schematic pictorial illustration of a system for measurement of body dimensions, in accordance with an embodiment of the invention;

FIG. 2 is a schematic pictorial illustration of an imaging module attached to a smartphone, in accordance with an embodiment of the invention;

FIGS. 3A and 3B are schematic front and rear pictorial views of an imaging module for attachment to a smartphone, in accordance with an embodiment of the invention;

FIG. 4 is a schematic frontal view of a calibration cap for an imaging module, in accordance with an embodiment of the invention;

FIG. 5 is a flow chart that schematically illustrates a method for measurement of body dimensions, in accordance with an embodiment of the invention; and

FIG. 6 is a schematic representation of a user interface screen of a body measurement application, in accordance with an embodiment of the invention.

### DETAILED DESCRIPTION OF EMBODIMENTS

### Overview

There is a growing demand among consumers for accurate, convenient measurement of their body dimensions. For example, in on-line shopping applications, such measurements enable consumers to choose clothing whose size and style will fit them well, thus enhancing customer satisfaction and reducing the percentage of garments that are returned after purchase. As another example, people who have undertaken personal fitness programs have an interest in tracking the resulting changes in their body size and shape over time, such as reduction in their waist and hip circumferences, as well as increases in muscle size.

Embodiments of the present invention that are described herein address these needs by providing novel garments, cameras and software that facilitate such measurements. In the disclosed embodiments, a garment of this sort comprises an elastic fabric having a predefined pattern extending across its surface thereof. The garment fits snugly over a part of the body that is to be measured, such as a shirt, bra, or leggings, so that the clastic fabric stretches across the part of the body.

A camera captures images of the pattern while contacting and traversing across the surface of the fabric while the subject wears the garment. In this manner, the images are captured at a fixed distance from the surface of the garment, and thus at a known ratio of image pixels to units of distance. This mode of operation of the camera obviates inaccuracies that commonly arise in non-contact measurement. The user simply slides the camera across the garment in order to capture images of the pattern at a sufficient number of locations. The local deformation of the pattern on the garment at each location is indicative of the degree of stretching of the fabric, and thus of the underlying body dimensions. Some embodiments provide an imaging module comprising a special-purpose housing, which fits over a mobile electronic device, such as a smartphone, and enables the existing image sensor of the mobile electronic device to capture images of the garment in this manner.

One or more processors process the images captured by the camera so as measure the local deformations. The measurement may be made locally, for example by an application running on a processor embedded in the user's smartphone or other mobile electronic device. Alternatively or alternatively, image data may be transmitted over a network to a server for these purposes; or the processing may be performed in collaboration between one or more local devices and the server. In any case, the processor responsible combines the measurements of pattern deformation at different locations on the garment in order to compute one or more dimensions of the part of the body over which the garment is worn. The application then, for example, outputs an indication of the dimensions to the user in the form of body measurements and/or a 3D avatar, or in the form of a recommendation of an article of clothing to purchase, or a comparison of the current dimensions to earlier, stored values.

Other apparatus, garments, methods of operation, and software for performing these sorts of measurements are described in the above-mentioned PCT Patent Application PCT/IB2020/052526. The image-based measurement techniques that are described herein may be combined with other measurement methods and devices that are known in the art, such as those described in the above-mentioned PCT International Publication WO 2015/181661, for purposes of enhancing accuracy and versatility of measurement.

### System Description

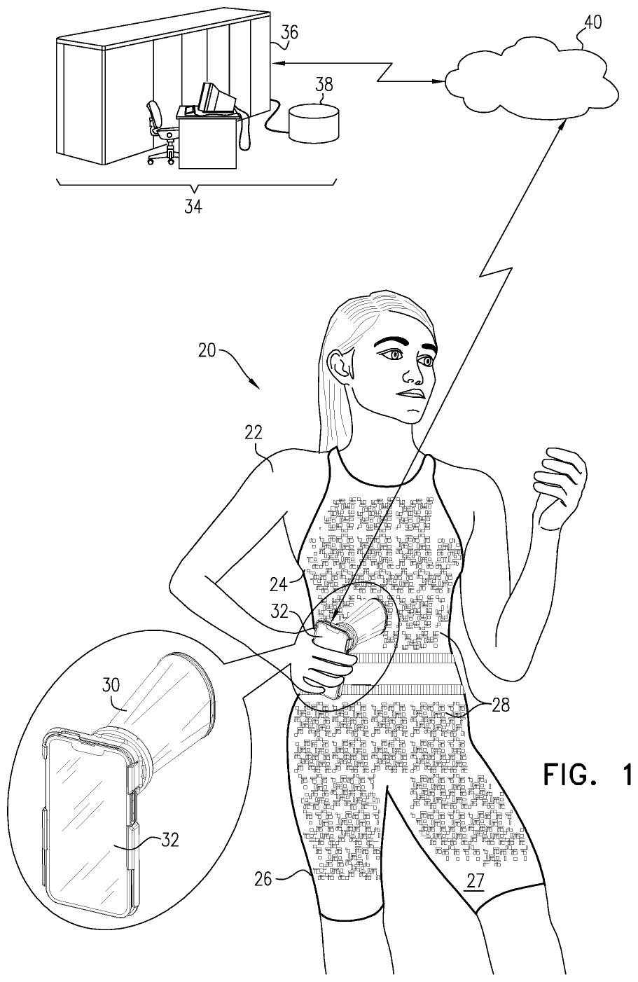

FIG. 1 is a schematic pictorial illustration of a system 20 for measurement of body dimensions, in accordance with an embodiment of the invention. A subject 22 wears garments 24, 26 comprising an elastic fabric 27 over her torso, abdomen, and legs. Garments 24, 26 fit sufficiently snugly so that the fabric stretches across these parts of the subject's body. Any suitable clastic fabric can be used for this purpose, with any desired degree of stretch, from as little as 6% to as much as 300% or more, depending on design and application requirements. Different parts of the same garment may have different degrees of stretch.

A pattern 28 extends across the surface of fabric 27 and deforms as the shape of the body stretches the fabric. Details of this process, as well as patterns that may be used on garments in system 20, are described in the above-mentioned PCT Patent Application PCT/IB2020/052526. Although pattern 28 may appear in FIG. 1 to be uniformly periodic, and thus to repeat itself over the area of garments 24 and 26, in practice it can be advantageous that the pattern be non-uniform and possibly non-repeating. Specifically, in some embodiments, the pattern comprises graphical symbols that are disposed at different, respective locations across the surface of fabric 27 and encode information identifying the respective locations and possibly additional information, for example about the garment type and model. Examples of symbols that can be used for this purpose include one- and two-dimensional barcodes, including QR codes, as well as ArUco markers, such as the markers described by Garrido-Jurado et al., in “Automatic generation and detection of highly reliable fiducial markers under occlusion,” published in Pattern Recognition 47(6), pages 2280-2292 (June 2014).

Subject 22 slides a smartphone 32 with an imaging module 30 across the surface of fabric 27 of garment 24 and/or 26. Imaging module 30 comprises a housing that attaches the imaging module to smartphone 32, as illustrated in the figures that follow. The camera module in smartphone 32 captures images of pattern 28 via imaging module 30 as the front end of the imaging module contacts and traverses across the surface of the fabric. In some embodiments, the processor in smartphone 32 processes the images to decode and measure the deformation of pattern 28. Additionally or alternatively, smartphone 32 may transmit the images and/or data extracted from the images to a remote computer, such as a server 34, for example via a wireless connection such as a cellular or Wi-Fi link.

In the pictured embodiment, smartphone 32 transmits image data to server 34 for further processing. Server 34 comprises a processor 36 and a memory 38, and communicates with smartphone 32 over a network 40, such as the Internet. For example, smartphone 32 may extract certain parameters from the images captured by the camera module in the smartphone and may then transmit the parameters via network 40 to server 34. Additionally or alternatively, smartphone 32 may transmit raw images of pattern 28 to server 34 for processing.

In an embodiment of the invention, some or all of the processing described herein is performed by the processor in smartphone 32 (or on another local computing device), using a suitable application running on the smartphone. For example, a processor in smartphone 32 may process the raw images of pattern 28, detect information encoded in the pattern, and measure the deformation of the pattern. Smartphone 32 may send this processed information to server 34.

In any case, the images captured by the camera module in smartphone 32 via imaging module 30 at multiple locations on the surface of fabric 27 are processed so as measure the local deformation of pattern 28 at each location due to stretching of the fabric, and then to compute, on the basis of the measured deformation, one or more dimensions of the parts of the body that are covered by garments 24 and/or 26.

System 20 is shown in FIG. 1, for the sake of concreteness and clarity, as comprising certain types of garments 24, 26, imaging module 30, and processing components. Alternatively, the principles of the present invention may be implemented using types of garments and cameras, as well as other processing components and system functionalities. All such alternative implementations are considered to be within the scope of the present invention.

### Imaging Modules

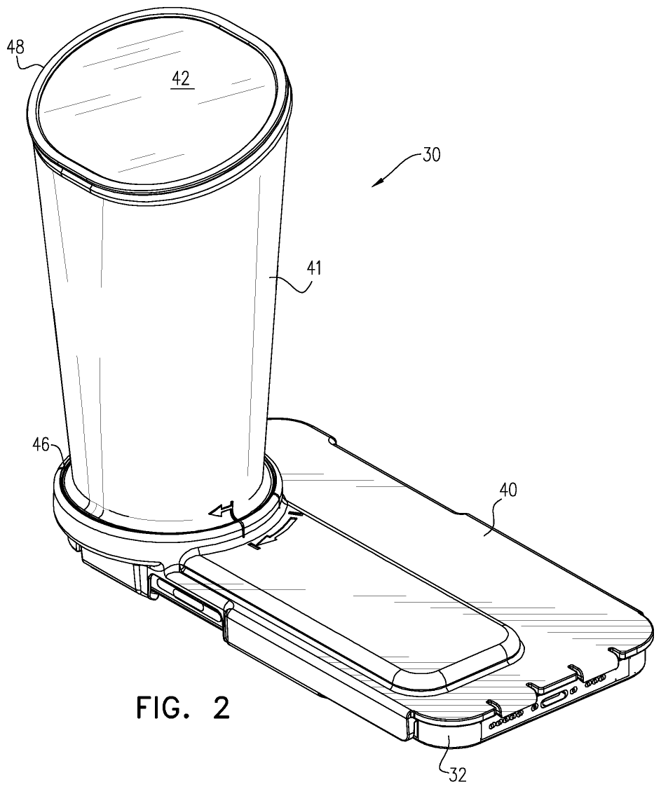

FIG. 2 is a schematic pictorial view of an imaging module 30, which is attached to a smartphone 32, in accordance with an embodiment of the invention. In this embodiment, the image sensor used to capture images of fabric 27 of garments 24 and 26 is the rear image sensor in the camera module that is built into smartphone 32. Imaging module 30 comprises a conical extension 41, which is attached to a housing 40 that fits over smartphone 32 and acts as a fastener to attach the imaging module to the smartphone. The term “housing” is used in the present description and in the claims to refer to a covering that fits around at least four sides of the mobile electronic device to which it is attached, for example, the back of smartphone 32 and the top, right, and left edges of the smartphone in the pictured example. Alternatively, other sorts of fasteners can be used, such as the sorts of fasteners shown in the above-mentioned PCT Patent Application PCT/IB2020/052526.

When housing 40 is fitted over smartphone 32, a rear opening 46 at the narrow end of conical extension 41 fits around the lens of the rear image sensor in the smartphone. Housings of different sizes and shapes can be provided to fit different models of smartphones. The back light of smartphone 32 may be operated to illuminate the fabric contacted by imaging module 30. Alternatively or additionally, conical extension 41 may be transparent or semitransparent to allow light to pass into the imaging module and illuminate pattern 28.

A front opening 48 of conical extension 41, which is larger than rear opening 46, is covered by a transparent window 42, which contacts and presses against fabric 27 as subject 22 slides imaging module 30 across the garment. Front opening 48 is cut at a bias, so that window 42 is oriented at an angle that is not parallel to the plane of rear opening 46 and of housing 40. Window 42 is thus non-perpendicular to the optical axis of the camera module in the smartphone. For example, window 42 is tilted by at least 50 relative to the plane of rear opening 46. This configuration is advantageous in reducing specular reflections from the back light of the smartphone into the camera module.

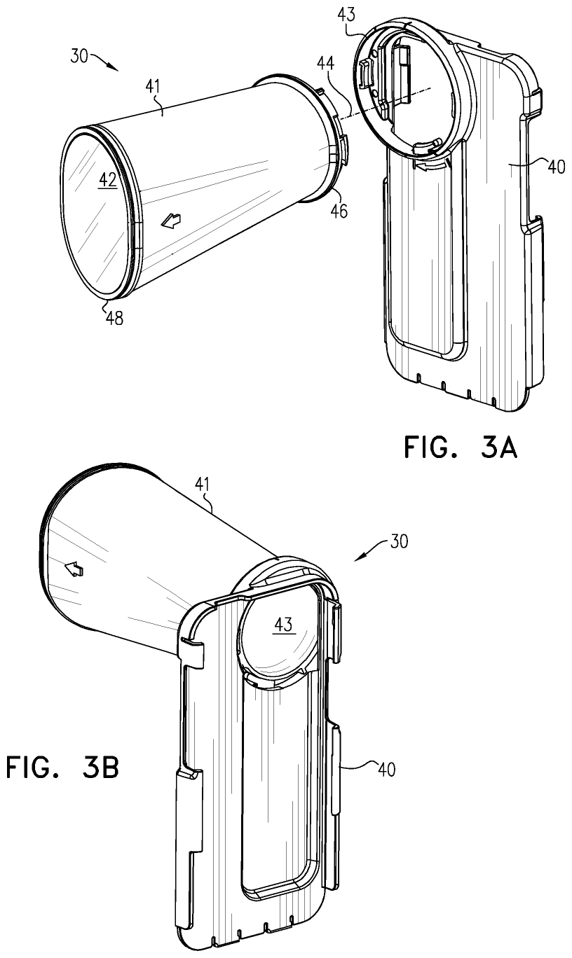

FIGS. 3A and 3B are schematic front and rear pictorial views of imaging module 30, in accordance with an embodiment of the invention. Rear opening 46 of conical extension 41 is aligned with an aperture 43 in housing 40, which opens over the camera module of the smartphone. In this embodiment, conical extension 41 attaches to housing 40 by a rotatable locking connection, thus allowing the same conical extension to be used with multiple different smartphone models. Alternatively, the conical extension may be integrally formed with the housing or otherwise permanently fixed to the housing. Housing 40 may comprise any suitable material, such as plastic or rubber. In one embodiment, housing 40 is designed to slide on and off over the smartphone.

The term “conical” is used in the context of the present description and the claims to refer to the shape of a hollow object having a cross-section that widens gradually along an axis 44 from a narrow end to a wide end of the object. The cross-section may be circular, or it may alternatively have any other suitable shape, such as the oblong shape of conical extension 41.

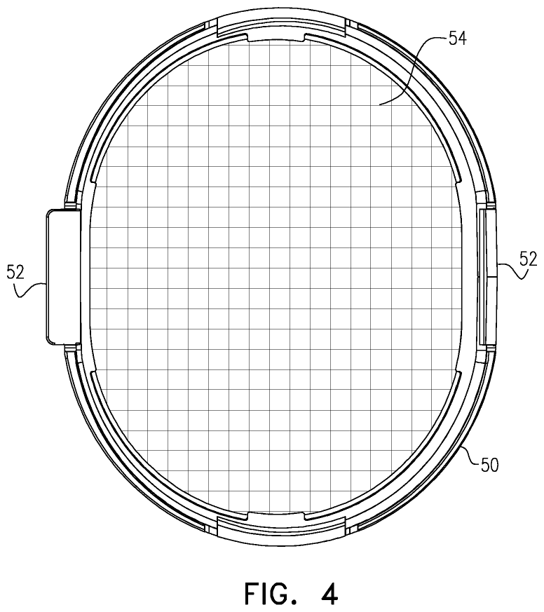

FIG. 4 is a schematic frontal view of a calibration cap 50 for imaging module 30, in accordance with an embodiment of the invention. To measure accurately the deformation of pattern 28 on fabric 27 and maintain consistency between measurements taken at different times, it is important that the magnification of the images captured by the camera module in smartphone 32 be calibrated. For the purpose of calibration, cap 50 is fitted over window 42 and is held in place, for example by clips 52. In this configuration, the camera module captures images of a reticle grid 54 on the inner surface of cap 50. The processor in smartphone 32 processes these images to calibrate the magnification, including compensation for the angular bias of window 42.

Alternatively, cap 50 may have other sorts of patterns. Further alternatively or additionally, fiducial marks on window 42 itself and/or on the inner surface of the cone of imaging module 30 may be used in calibrating the magnification.

### Methods of Operation

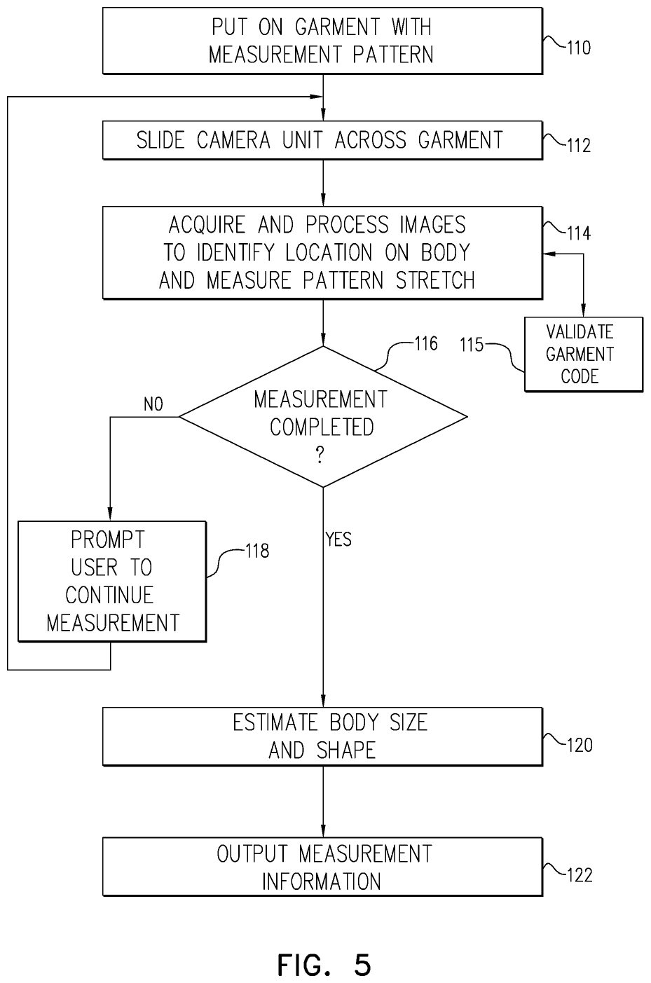

FIG. 5 is a flow chart that schematically illustrates a method for measurement of body dimensions, in accordance with an embodiment of the invention. The method is described here, for the sake of convenience and clarity, with reference to the elements of system 20, as shown in FIGS. 1 and 2. Alternatively, the method may be carried out using any other suitable sorts of cameras and garments that satisfy the criteria set forth herein. The term “user” in the description below may refer either to subject 22 herself or to another user assisting the subject in the measurement process. Although FIG. 1 and the present language refer to a female subject, the principles of the present embodiments may equally apply to male subjects.

To initiate the present method, subject 22 puts on a garment with a measurement pattern, such as garment 24 and/or 26 at a dressing step 110. The user (i.e., the subject herself or an assisting user) fits imaging module 30 over smartphone 32 and opens the measurement application on the smartphone. At this stage the user may optionally put the calibration cap 50 onto imaging module 30 and use the measurement application to calibrate the smartphone camera. Calibration cap 50 can be used before each measurement session and/or each time imaging module 30 is put onto smartphone 32 and/or from time to time. The user then slides imaging module 30 across the surface of garment 24, at an image capture step 112. It can be advantageous that subject 22 stand still at this step, or move as little as possible.

The measurement application actuates the camera in smartphone 32 to capture a continuous series of image frames, each showing a part of pattern 28. The processor in smartphone 32 processes the images under the control of the measurement application. Alternatively or additionally, the images may be processed remotely, for example by processor 36 in server 34.

The processor processes the images captured at step 112 in order to identify the location on garment 24 and/or 26 at which each image was acquired and measure the pattern deformation in the image, at an image processing step 114. In the description that follows, it will be assumed that step 114 is carried out by the processor in smartphone 32. Alternatively, however, step 114 may be carried out in whole or in part by server 34. Furthermore, although step 114 is shown in FIG. 5 as following step 112, in practice these steps may be carried out concurrently, with the processor in smartphone 32 processing a certain image at the same time as the camera module acquires a subsequent image.

As the user slides imaging module 30 over garment 24 and/or 26, it is desirable that window 42 be pressed tightly against fabric 27 to ensure good measurement accuracy. To verify that window 42 is pressed tightly against the fabric, the measuring application turns off the light of smartphone 32 during step 114 for an interval of one or several image frames. The processor then verifies that the image frames captured during this interval are dark, hence indicating that window 42 is being pressed tight against fabric 27. If not, the processor prompts the user to press the window more firmly against the fabric.

In the course of step 114, the processor extracts the location at which each image was captured. The location can be identified on the basis of the unique local pattern that appears in the image, such as a graphical symbol that is indicative of the location. For example, in some embodiments, pattern 28 comprises codes, such as QR codes or ArUco codes, which encode values that are indicative of the location. In this case, the processor decodes the code while also measuring the deformation of the code. The application running on smartphone 32 converts the decoded value to a location on garment 24 and/or 26, either based on data stored on smartphone 32 or by submitting a query to server 34. The measured deformation of the detected codes can be used in reconstructing the 3D shape of subject 22.

The processor identifies features in the pattern 28 in each image, such as edges and corners, and computes the distances between these features in order to measure the deformation of the pattern due to stretching of fabric 27. For example, the processor may ascertain that a certain pair of features are now 8 mm apart, whereas the baseline distance between these features, before garment 24 was stretched over the user's body, was only 4 mm.

Before proceeding with the measurements and further interaction with the user, the processor in smartphone 32 may read and decode one or more symbols in pattern 28 on the garment, and may process the decoded values in order to verify that the garment is authentic, at a validation step 115. To enable such validation and authentication, pattern 28 on garment 24 includes, in some embodiments, an identification code at a predefined location, and the user may be prompted to slide imaging module 30 over this location at some stage in step 112. Alternatively, all of the symbols in pattern 28 may encode the identification code, along with location information. The processor in smartphone 32 then validates the identification code, for example by checking the value of the code in memory 38 of server 34, before proceeding with further measurements in step 114.

Assuming authentication (if necessary) is completed successfully at step 115, the camera module and processor continue acquiring images and measuring pattern stretch at step 114, as the user continues sliding imaging module 30 across the garment. The processor in smartphone 32 checks the locations of the acquired parts of the pattern at a completion checking step 116. If there are still significant parts of garment 24 and/or 26 that have not been scanned, the processor prompts the user to continue the measurements, at a user prompting step 118. For example, the processor may issue a message via the user interface of smartphone 32 indicating the area of garment 24 that have not yet been scanned.

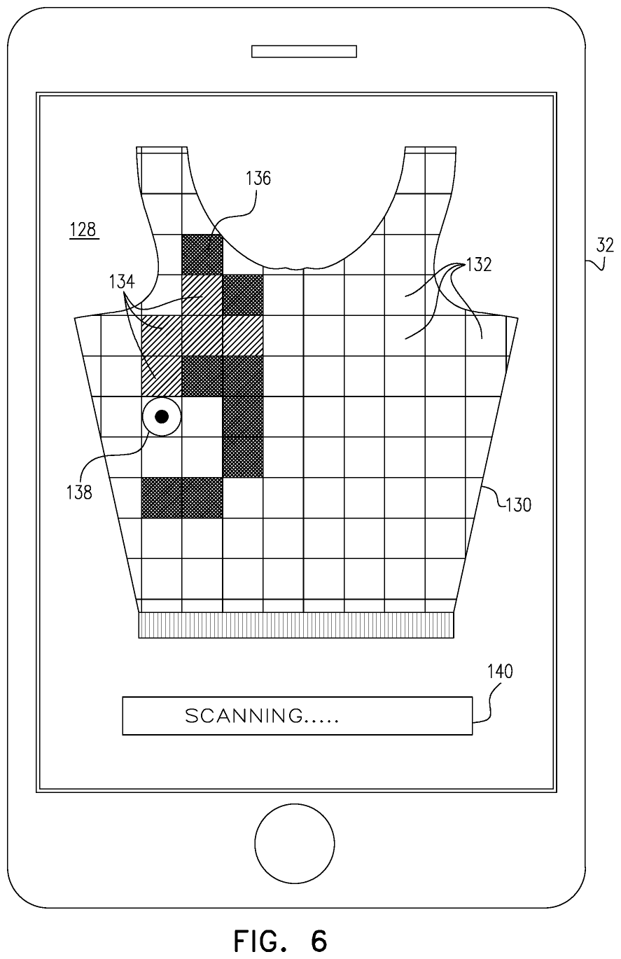

In one embodiment, the processor prompts the user to scan each location on the garment twice and compares the results to verify that they are accurate. This approach is useful in improving measurement accuracy and rejecting artifacts. A user interface screen for this purpose is shown in FIG. 6.

When the processor in smartphone 32 finds at step 116 that the measurements have been completed, it proceeds to compute one or more dimensions of the part of the body of subject 22 that is covered by garment 24 and/or 26, at a dimension computation step 120. More generally, the measurements that the processor has made of the local deformation of the pattern enable it to estimate and model the entire size and shape of this part of the subject's body. Alternatively, the user may decide to end the scan, whereupon the dimensions are computed on the basis of the measurements that have been made.

As another alternative, the processor may compute and dimensions of the body of subject 22 in parallel with step 114, based on the cumulative measurements that have been made at each point in the process. For example, the user may first run imaging module 30 around the body, giving measurements of deformation that will enable the processor to estimate the circumference of the body at each of a series of longitudinal coordinates. The processor may display these estimates and prompt the user to capture images at additional locations in order to fill in details of the body shape.

When step 120 has been completed, the processor in smartphone 32 outputs an indication of the computed dimension or dimensions to the user, at an output step 122. As noted earlier, the application running on smartphone 32 may first validate the identification code of garment 24 and/or 26 at step 115 before providing this output. The output at step 122 may present the body shape and size information in various forms, for example by showing a 3D avatar of the body, by presenting measurements of specific body parts, and/or by comparing values from different measuring sessions.

Specifically, when smartphone 32 or server 34 stores past values of the computed dimensions, the processor can compare the value of the dimension measured at step 120 to the stored values, and then provide the user with an indication of the change of the current dimension relative to the stored values. For example, the processor may display a trend illustrating the decrease is waist and hip size or increase in muscle size over the course of a program of exercise and/or weight loss. As another example, the changes in body dimensions can be reported to and used by a medical caregiver or physiotherapist for diagnosis and follow-up of the subject's medical and physiological condition.

Additionally or alternatively, the processor may use the computed dimensions in identifying one or more articles of clothing of a size suitable to be worn by subject 22, for example via an Internet shopping site. Methods for on-line shopping that can be applied using dedicated measurement garments, such as garments 24 and 26, are described, for example, in U.S. Pat. No. 9,858,611, whose disclosure is incorporated herein by reference. Additionally or alternatively, server 34 may match groups of users having similar body dimensions and may enable these users to exchange information and recommendations regarding clothing that they have found to fit them well.

FIG. 6 is a schematic representation of a user interface screen 128 of a body measurement application running on smartphone 32, in accordance with an embodiment of the invention. Screen 128 may be displayed at step 118 and/or step 114 in FIG. 5 in order to track the locations on the surface of fabric 27 at which imaging module 30 has captured images, and to prompt the user on this basis to shift the camera to areas of garment 24 and/or 26 in which images of pattern 28 have not yet been captured.

In this example, screen 128 presents a graphical representation 130 of the garment, with areas 132 marked to indicate whether or not they have been scanned by the camera. Initially, all areas 132 are unmarked. As the user slides imaging module 30 over garment 24, areas 134 are marked in a certain color to indicate that they have been scanned once, and then areas 136 are marked in a different color to show that they have been scanned twice. The processor in smartphone 32 compares the measured values of pattern deformation in each of the two scans in ach area 132 and marks areas 136 when the values match to within a predefined tolerance. (Otherwise, the processor prompts the user to make an additional measurement at the location in question.) Alternatively, color indications may be used to highlight areas that were not scanned yet, scanned once, or scanned twice and are within tolerance, for example, using red, yellow and green as in a traffic light. In addition, areas that were scanned twice and are within tolerance might lock, so their value will not change even if the areas are scanned again during the measuring session. A special mark 138 shows the current location of imaging module 30. A completion bar 140 may give an indication of the fraction of the area of garment 24 that has been covered.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Measurement apparatus, comprising:
a garment comprising an elastic fabric having a predefined pattern extending across a surface thereof and configured to be worn over a part of a body of a subject, such that the elastic fabric stretches across the part of the body; and
an imaging module, comprising:
a fastener configured for attachment to a mobile electronic device having a camera module; and
a conical extension having a rear opening, which is configured to be held by the fastener in alignment with the camera module, and a front opening, which is larger than the rear opening and is configured to contact and traverse across the fabric, whereby the camera module captures through the front opening images that are indicative of local deformations of the pattern of the fabric.

2. The apparatus according to claim 1, and comprising a processor, which is configured to process the images captured by the camera module at multiple locations on the surface of the fabric so as measure a local deformation of the pattern at the multiple locations due to stretching of the fabric, and to compute a dimension of the part of the body responsively to the measured deformation.

3. The apparatus according to claim 2, wherein the processor is contained in the mobile electronic device.

4. The apparatus according to claim 2, and comprising a cap, having a calibration pattern formed thereon, which is configured to fit over the front opening of the conical extension, wherein the processor is configured to calibrate a magnification of the camera by processing an image of the calibration pattern captured by the camera module and to apply the calibrated magnification in measuring the local deformation of the pattern.

5. The apparatus according to claim 2, wherein the processor is configured to output an indication of the computed dimension to a user of the apparatus.

6. The apparatus according to claim 1, wherein the mobile electronic device is a smartphone, and wherein the fastener comprises a housing configured to fit over the smartphone.

7. The apparatus according to claim 1, wherein the pattern comprises multiple graphical symbols, which are disposed at different, respective locations across the surface of the fabric and encode information identifying the respective locations.

8. The apparatus according to claim 1, wherein the imaging module comprises a transparent window covering the front opening of the conical extension so as to contact the surface of the fabric.

9. The apparatus according to claim 8, wherein the window is oriented at an angle that is not parallel to a plane of the rear opening of the conical extension.

10. An imaging module, comprising:
a housing configured to fit over a smartphone; and
a conical extension having, at a rear end of the conical extension, a rear opening, which is configured to be held by the housing in alignment with a camera module of the smartphone, and having, at a front end of the conical extension, opposite the rear end, a front opening, which is larger than the rear opening.

11. The module according to claim 10, and comprising a transparent window covering the front opening of the conical extension.

12. The module according to claim 11, wherein the window is oriented at an angle that is not parallel to a plane of the rear opening of the conical extension.

13. The module according to claim 10, and comprising a locking connection, which attaches the conical extension to the housing.

14. The module according to claim 10, wherein the conical extension is permanently fixed to the housing.

15. The module according to claim 10, and comprising a cap, having a calibration pattern formed thereon, which is configured to fit over the front opening of the conical extension.

16. A method for measurement, comprising:
providing a garment comprising an elastic fabric having a predefined pattern extending across a surface thereof and configured to be worn over a part of a body of a subject, such that the elastic fabric stretches across the part of the body;
while the subject is wearing the garment, capturing images of the pattern using a camera, while the camera contacts and traverses across the surface of the fabric;
processing the images captured by the camera at multiple locations on the surface of the fabric so as measure a local deformation of the pattern at the multiple locations due to stretching of the fabric; and
outputting to a user an indication of the locations at which the images were captured.

17. The method according to claim 16, and comprising computing a dimension of the part of the body responsively to the measured deformation, and outputting an indication of the computed dimension to the user.

18. The method according to claim 16, wherein capturing the images comprises fitting an imaging module over a mobile electronic device, the imaging module comprising a conical extension having a rear opening aligned with a camera module of the mobile electronic device and a front opening, which is larger than the rear opening and is configured to contact and traverse across the fabric, whereby the camera module captures the images through the front opening.

19. The method according to claim 16, wherein the predefined pattern comprises multiple graphical symbols, which are disposed different, respective locations across the surface of the fabric and encode information identifying the respective locations, and wherein processing the images comprises decoding the information.

20. The method according to claim 16, wherein processing the images comprises tracking the locations on the surface of the fabric at which the camera captures the images, and wherein outputting the indication comprises prompting a user, responsively to the tracked locations, to shift the camera to an area of the garment in which the images of the pattern have not yet been captured.

21. The method according to claim 20, wherein prompting the user comprises directing the user to shift the camera so as to traverse at least some of the locations multiple times, whereby the camera captures at least first and second images of each of the at least some of the locations, and wherein processing the images comprises comparing the first and second images so as to verify an accuracy of measurement of the local deformations.

22. The method according to claim 21, wherein directing the user comprises presenting the locations on a display, and marking the locations on the display to indicate a number of times that each of the locations has been traversed.

23. The method according to claim 16, wherein capturing the images comprises illuminating an area contacted by the camera, and wherein the method comprises capturing one or more image frames while the area is not illuminated, and verifying that the one or more image frames are dark as an indication that the camera is in contact with the surface of the fabric.
